(19) United States
(12) Patent Application Publication
(10) Pub. No.: US 2008/0268492 A1
(43) Pub. Date: Oct. 30, 2008

(54) Methods for Determining Optimal Techniques for Vitrification of Isolated Cells

(75) Inventors: Steven Francis Mullen, Fontana, CA (US); John K. Critser, Columbia, MO (US); Zi-Jiang Chen, Broadview Heights, OH (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: THE CURATORS OF THE UNIVERSITY OF MISSOURI, Columbia, MO (US)

(21) Appl. No.: 12/101,880

(22) Filed: Apr. 11, 2008

Related U.S. Application Data
(60) Provisional application No. 60/923,153, filed on Apr. 12, 2007.

(51) Int. Cl. C12Q 1/02

(52) U.S. Cl. 435/29

(57) ABSTRACT

A method to optimize a vitrification procedure for suspended cells uses factors such as the physical properties of solutions, the cell permeability to water and permeable cryoprotectants, and the osmotic tolerance of the cells to identify a method to minimize several stresses associated with vitrification procedures.

### CROSS-REFERENCE TO RELATED APPLICATIONS

The present application claims priority to co-pending provisional application No. 60/923,153, filed on Apr. 12, 2007, entitled “A General Method for Determining an Optimal Technique for Vitrification of Isolated Cells”, the disclosure of which is incorporated herein in its entirety.

### BACKGROUND

The present disclosure relates to the field of preservation of cells, and particularly to systems and methods for cryopreservation of cells.

Isolated cells collected from body fluids (e.g. blood, semen) or from tissues have relatively finite life spans. However, it is often desirable to preserve such cells for future use (days, months, or even years). In order to maintain the cell viability, the cells usually have to be held in a state such that the metabolism is significantly reduced, or even stopped. Preservation methods for maintaining cell viability for more than a few days usually rely upon cooling the cells to low sub-zero temperatures.

Temperatures used for long-term storage of cells are typically below the glass transition temperature of water (˜140 K). At such temperatures, the water of dilute aqueous solutions (including the cytoplasm of cells) is thermodynamically stable in the crystalline form (i.e., ice). However, the formation of ice inside of cells (hereafter referred to as intracellular ice formation; IIF) is usually lethal to cells. Therefore, avoiding IIF is an important consideration when designing cryopreservation methods.

Intracellular ice formation can be avoided by increasing the solute concentration of the cytoplasm to the point where it will vitrify during cooling to the storage temperature and warming. (cf., Fahy G M, MacFarlane D R, Angell C A and Meryman H T (1984) “Vitrification as an approach to cryopreservation”, Cryobiology 21, 407-426). This can be done in one of two ways. The first involves cellular dehydration during slow cooling as a result of extracellular ice formation and the resulting driving force for exosmosis. (Gao D, Mazur P and Critser J K (1997) “Fundamental Cryobiology of Mammalian Spermatozoa”, in Karow, A M and Critser, J K (eds) “Reproductive Tissue Banking, Scientific Principles”, Vol Academic Press, San Diego, pp. 263-328). The second involves replacing cytoplasmic water with solutes that promote vitrification. (MacFarlane D R and Forsyth M (1990) “Recent insights on the role of cryoprotective agents in vitrification”, Cryobiology 27, 345-358).

In general, solutions used to cryopreserve cells contain solutes that confer protection to the cells during cryopreservation. The concentration of these solutes in the solution varies depending upon the method of cryopreservation utilized. For example, slow-cooling methods usually use solutes at concentrations between 1 and 2 molar. However, such a low concentration of solutes will not prevent ice formation during cooling and warming when traditional cryopreservation devices are employed. In order to avoid ice formation altogether at typical cooling and warming rates used in vitrification procedures (typically between 1×10³ and 1×10⁴° C./min) solute concentrations need to be much higher (˜6 to 7 mol/L). Exposing cells to such solutions can be damaging for several reasons. First, the high concentration of solutes can have direct chemical toxicity on the cells (Fahy G M (1986) “The relevance of cryoprotectant “toxicity” to cryobiology”, Cryobiology 23, 1-13; Fahy G M, Lilley T H, Linsdell H, Douglas M S and Meryman H T (1990) “Cryoprotectant toxicity and cryoprotectant toxicity reduction: in search of molecular mechanisms”, Cryobiology 27, 247-268). However, exposing cells to solutions with high osmolalities such as these can also cause osmotic shock and cell death. Fortunately, these forms of damage can be controlled to some degree. For example, compounds that permeate the cell can be chosen that have relatively low toxicity. Additionally, permeating compounds can be replaced to some degree by non-permeating compounds, which can reduce the chemical toxicity of these solutions even further. Osmotic damage can be controlled by exposing cells to solutions in a stepwise manner, with the total concentration of solutes increasing gradually in each of the different solutions.

In these prior approaches, the potential for cell damage exists. Therefore, it is important to determine the appropriate combination of the many variables in order to avoid cell damage. For instance, cells that are intolerant of exposure to sub-physiologic temperatures for more than brief periods of time (i.e., seconds to minutes) require the use of fast cooling methods (i.e. vitrification). Currently, the choice for a vitrification solution is usually conducted by making general assumptions about how concentrated the solutes in a solution need to be in order to maintain a vitreous state during cooling and warming. For example, it is common for experimenters to choose solutes at concentrations in increments of 10% or 0.5 M for testing. However, with the use of techniques such as differential scanning calorimetry combined with crystallization theory (Baudot A and Odagescu V (2004) “Thermal properties of ethylene glycol aqueous solutions”, Cryobiology 48, 283-294), the total concentration of solutes in a solution necessary to maintain a vitreous state can be estimated more precisely for any cooling and warming rate.

The method to expose cells to such solutions is often chosen in a similar manner. For example, if a solution with 40% ethylene glycol is chosen as the final vitrification solution, the procedure to expose cells to this solution is usually done in a stepwise manner, with each step having a fractional percent of the final solution as the choice (i.e., 20% for the first step, then 40% for the second). Furthermore, the amount of time for which each step proceeds is usually chosen without consideration for the time it takes the compound to enter the cell. However, one can use a more systematic approach if the cell permeability to the components in question (i.e., water and permeating cryoprotectants) is accounted for and the tolerance of the cell to volume changes is also considered.

Previous approaches to optimize permeating compound addition to and removal from cells have been disclosed in U.S. Pat. Nos. 5,691,133 and 5,595,866 to Critser et al., the disclosure of which is incorporated herein by reference. These patents describe methods to add and remove permeating cryoprotectants for spermatozoa by taking into account the osmotic tolerance and membrane permeability characteristics of the cells. However, they do not disclose a method for choosing an optimal combination of cryoprotectants to use for vitrification.

These methods have been modified by approaches disclosed in U.S. Pat. Nos. 5,700,632, 5,753,427, and 5,776,769 to Critser et al., the disclosures of which are incorporated herein by reference, which describe the use of a device containing a permeable membrane which allows solutions to be exchanged easily between steps and developing more extensive mathematical models to optimize cryoprotectant addition to and removal from cells.

Other methods to optimize vitrification, particularly for oocytes, have been disclosed. These methods generally rely upon methods and devices to increase the rate of cooling and warming during vitrification. For example, U.S. Pat. No. 6,982,172 to Yang et al., the disclosure of which is incorporated herein by reference, discloses the application of the “solid surface vitrification” technique for bovine oocytes. This disclosure relies upon the use of a very specific vitrification solution and method to achieve rapid cooling, namely the deposition of a small drop of medium containing the cells onto a pre-cooled surface, allowing rapid cooling to take place. U.S. Pat. No. 6,916,602 to Arav, the disclosure of which is incorporated herein by reference, describes a method to preserve a whole organ (ovary) for future utilization based upon a very specific method for cryopreservation. U.S. Pat. No. 5,985,538 to Stachecki, the disclosure of which is incorporated herein by reference, discloses a method to reduce the potential toxicity of a cryopreservation solution used in a slow-cooling method by reducing the concentration of sodium ions in the freezing medium.

U.S. Pat. No. 7,087,370 to Forest et al., the disclosure of which is incorporated herein by reference, describes a vitrification kit that contains a vitrification solution and “transfer instrument” (e.g. nylon loop) which allows rapid cooling to occur during specimen transfer to liquid nitrogen. U.S. Pat. No. 6,921,633 to Baust et al, the disclosure of which is incorporated herein by reference, describes a very general means to achieve vitrification by using highly concentrated solutions and specific molecular inhibitors of programmed cell death.

U.S. Pat. No. 4,559,298 to Fahy, the disclosure of which is incorporated herein by reference, describes a method to achieve vitrification of biological material in a method which takes into consideration the potential toxic properties of solutes and proposes the replacement of permeable solutes with non-permeable solutes. In addition, this patent describes exposure of the biomaterial to the vitrification solutions at reduced temperatures to further alleviate potential chemical toxicity, as well as the application of pressure to facilitate vitrification as well as to minimize the potential of devitrification during warming. U.S. Pat. No. 5,723,282 to Fahy et al., the disclosure of which is incorporated herein by reference, describes a mechanical device to perfuse organs with cryoprotectant solutions. U.S. Pat. No. 5,821,045 to Fahy et al., the disclosure of which is incorporated herein by reference, describes improvements to the mechanical perfusion of cryoprotectants into organs for vitrification.

None of these prior disclosed techniques, address optimization of cryoprotectant solutions or processes. There is a need for a method that facilitates selection of a particular approach that is best suited for any particular temperature sensitive cells.

### SUMMARY

The present invention relates to a method for determining an optimal approach for the vitrification of cells in suspension. The method relates to identifying a solution that contains a combination of permeating and non-permeating cryoprotective compounds. The combination is determined to be optimal if it contains the minimum amount of permeating compounds in relation to the total solute concentration that is necessary to maintain a vitreous state throughout a cryopreservation procedure. Minimizing the permeating solute concentration should result in the minimum chemically toxic effects. The amount of non-permeating solute is also optimal, as its concentration is chosen such that the overall effects on the cell volume of such a solution are kept within predefined tolerable limits.

In accordance with certain aspect of the invention, once an ideal vitrification solution is chosen, the next step involves determining the appropriate means to load the cells in question with the permeating solute. This can be a critical step, as the concentration of permeable solute remains high despite the use of some non-permeable solute. Transferring a cell directly to the solution used to vitrify the cells may result in excessive osmotic perturbations such as a drastic reduction in cell volume. Therefore, it is essential to determine a means to load the cell with the permeable solute without exceeding the tolerable level of osmotic stress. It is noted that in the context of the present disclosure, osmotic stress can be equated with cell volume changes. One step of the present inventive optimization process involves determining the osmotic tolerance of cells as measured by their cell volume changes.

Another step involves determining an optimal combination of permeating and non-permeating solutes to be included in a vitrification solution such that: 1) equilibrating cells with the solution does not result in them exceeding their osmotic tolerance volume limits as previously determined; and 2) the total solute concentration in the solution will maintain a vitreous state during cooling and warming. In a further step, a determination is made as to means to add and remove the permeating cryoprotectant from the cells in question without them exceeding their osmotic tolerance volume limits.

Certain aspects of the present invention relate to the determination of an optimal vitrification method based upon identifying a vitrification solution which has an optimal combination of permeating and non-permeating solutes. In accordance with one feature, an optimal combination may be defined as a combination that contains the maximum amount of non-permeating solute (hence the minimum amount of permeating solute which should minimize the chemical toxicity of the solution) such that: 1) the solution will maintain a vitreous state throughout the cooling and warming process; and 2) the effects of equilibrating a cell with such a solution will result in the cell volume being reduced just to the point that is defined as the lower osmotic tolerance limit. One feature of this invention resides in the determination of an optimal vitrification solution such that the combination of solutes accounts for the toxic properties of the solution (both osmotic and chemical) and the solution has the appropriate amount of dissolved solutes to maintain a vitreous state during cooling and warming.

### DESCRIPTION OF THE FIGURES

FIG. 1 is a graph of cell survival probabilities as a function of solution osmolality.

FIG. 2 is a graph of sucrose concentration as a ratio of total solute necessary to maintain a vitreous state of a cell during cooling and warming.

FIG. 3 includes graphs of heat flow as a function of temperature for various weight percent values for solution concentration.

FIG. 4 is a graph showing the effect of vitrification solutions on cell volume.

FIG. 5 includes a pair of graphs showing normalized cell volume changes over time during the addition and removal of ethylene glycol.

FIG. 6 is a table of solution parameters for a 4-step addition process according to one example of the present inventive method.

FIG. 7 is a table of solution parameters for a 2-step removal process according to one example of the present inventive method.

FIGS. 8A-D are photographic depictions of steps in determining cell volume using image analysis according to one embodiment of the methods disclosed herein.

FIG. 9 is a graph showing oocyte volume changes as a function of time and temperature when exposed to an EG containing solution.

FIG. 10 includes two Arrhenius plots of the relationship between temperature and two permeability parameters for oocytes.

### DESCRIPTION OF THE PREFERRED EMBODIMENTS

Specific language is used to describe this invention to promote an understanding of the invention and its principles. It must be understood that no specific limitation of the scope of this invention is intended by using this specific language. Any alteration and further modification of the described methods and any application of the principles of this invention are also intended that normally occur to one skilled in the art.

Vitrification is often described as the solidification of a liquid not by crystallization, but due to an extreme elevation of the viscosity of the solution as a result of a decrease in temperature (Fahy G M, MacFarlane D R, Angell C A and Meryman H T (1984) “Vitrification as an approach to cryopreservation”, Cryobiology 21, 407-426). As such, an aqueous solution that is in a vitrified state, by definition, does not contain ice crystals. The ability of an aqueous solution to maintain a true vitreous state differs depending upon the interaction of several variables, including solute concentration, solute type, and cooling/warming rates.

In general, the cooling and warming rates for a vitrification procedure are fixed as a result of the container in which the cell suspension is held. Therefore, other variables need to be modified in order to effect vitrification. It is a general principle that as the concentration of solutes in a solution increases, the cooling and warming rates necessary to ensure vitrification decreases. Hence, for a solution containing a combination of solutes, there will be a minimum concentration of solutes that can attain a vitreous state for a specific cooling and warming rate. It is also a general rule that a direct correlation exists between the concentration of solutes in a solution and the toxic properties of that solution to cells. Therefore, solutions with reduced solute concentrations are generally more tolerable to cells. Accordingly, one optimum selection is to choose the minimum solute concentration necessary to attain a vitreous state during cooling and warming when trying to vitrify cells if order to minimize the detrimental effects of the procedure.

A corollary is that the chemically toxic effect of solutes is more acute when the solute is inside rather than outside of the cell. Therefore, the replacement of permeating with non-permeating solutes is one general means by which the overall chemical toxicity of a solution may be reduced. However, non-permeating solutes have more damaging osmotic effects on cells than permeating compounds. Therefore, an optimal combination of permeating and non-permeating solutes should be achieved such that both the chemical and osmotic damage are minimized.

Based on the conclusion that an optimal combination of permeating and non-permeating solutes exists, an important aspect of optimizing vitrification methods is to determine this optimal combination in accordance with certain aspects of the present invention. Determining potential optimal combinations of permeating and non-permeating solutes first involves determining the appropriate proportions of each of these solutes in a solution such that the solution will vitrify. This can be done by holding the concentration of one of the solutes at a fixed level and varying the concentration of the other until a minimum concentration for the solute whose concentration is allowed to vary is found that will allow the maintenance of a vitrified state during cooling and warming. This process continues by changing the concentration of the fixed solute to a new value and repeating this process.

Once the combinations of solutes at the concentrations of interest that can maintain a vitreous state have been determined, the present invention involves determining which one of these combinations will be the best for use with the cells at hand. Determining the osmotic tolerance as measured by the cell volume change is generally accomplished by suspending cells in solutions containing non-permeating solutes of different osmolalities and determining both the cell volume response and the effect on viability. After this relationship has been established, the cell volume range that a chosen proportion of cells can tolerate is selected as the volume range within which the cells are to be maintained during the process of cryoprotectant loading and unloading.

To choose from the potential solutions to be used for the final vitrification solution, the solution is identified having the highest non-permeable to permeable solute ratio that will not result in the cell exceeding the tolerable volume range defined in the step described above. With such a choice, the permeable solute concentration can be reduced as much as possible, which should reduce the overall toxicity of the solution to the cells, yet not result in a high degree of osmotic damage. To determine the effect of the solutions on the cell volume, known equations can be used which describe the change in cell water volume and amount of permeable solute inside the cell. An optimum solution can thus be determined by summing the cell water volume, permeable solute volume (determined by multiplying the solute amount by the partial molar volume), and the volume of the cells occupied by solids (which can be determined from the known Boyle van't Hoff relationship describing the effect of non-permeable solute concentration on cell volume and extrapolating to infinite osmolality). The appropriate equations for each of these determinations can be found in standard texts and scientific papers on cell biology and fundamental cryobiology (see, e.g., Dick D A T (1979) “Structure and properties of water in the cell”, in Gilles, R (eds) “Mechanisms of osmoregulation in animals: Maintenance of cell volume”, Vol John Wiley and Sons, New York, pp. 3-45; Gao D, Mazur P and Critser J K (1997) “Fundamental Cryobiology of Mammalian Spermatozoa”, in Karow, A M and Critser, J K (eds) “Reproductive Tissue Banking, Scientific Principles”, Vol Academic Press, San Diego, pp. 263-328; Kleinhans F W (1998) “Membrane permeability modeling: Kedem-Katchalsky vs. a two-parameter formalism”, Cryobiology 37, 271-289).

After identifying an optimal solution, the next determination is of a method by which the permeating cryoprotectant can be loaded into the cell before vitrification, and unloaded from the cell after vitrification, without exceeding the tolerable cell volume range previously determined. This can also be done by solving known equations that describe changes in cell volume and intracellular cryoprotectant concentration, such as the calculations described in “Prevention of osmotic injury to human spermatozoa during addition and removal of glycerol”, Hum Reprod 10, 1109-1122 (Gao D Y, Liu J, Liu C, McGann L E, Watson P F, Kleinhans F W, Mazur P, Critser E S and Critser J K 1995), the disclosure of which is incorporated herein by reference.

Thus in accordance with the present invention a method is provided for identifying optimal combinations of solutes for inclusion in a vitrification solution and to identify optimal procedures to add and remove such solutes from cells without causing osmotic damage. This method comprises a first steps of determining an optimal combination of solutes in which the combination: i) contains a combination of permeable and non-permeable solutes such that the entire solution will maintain a vitreous state during cooling to cryogenic temperatures (<140 K) and warming from cryogenic temperatures; ii) contains concentrations of permeable solutes that can be tolerated by the cells; iii) contains the maximum amount of non-permeable solutes in relation to permeable solutes such that when the cell is allowed to come to equilibrium with the said solution, the cell volume will not be reduced below a level deemed tolerable to the cell population.

A subsequent step of the method involves determining an optimum method to load the permeable cryoprotectants into and unload the permeable cryoprotectants from the cells in a stepwise manner such that the cells are exposed to a solution containing the permeating cryoprotectants in a concentration that is more dilute than the concentration contained in the solution in which the cells are cooled. In a first step of this stepwise process, the total concentration of the initial solution is such that, when the cells are incubated in the solution, the cells will shrink osmotically just to the point of reaching a tolerable volume. After a predetermined amount of time, the cells are transferred in a second step of the stepwise process to a second solution containing the permeable cryoprotectants at a concentration higher than the first solution, but only at a concentration such that when the cells are transferred to the second solution the cells do not shrink below the cell volume deemed tolerable. In subsequent steps of the stepwise process the concentrations of cryoprotectants are increased until the point at which the cells can be transferred to the final solution used to vitrify the cells and the cells will equilibrate with the final solution and not shrink below the volume deemed tolerable.

This method can be applied where the cells consist of any isolated cell type. In certain embodiments, the permeable solutes can include any of the following components either singly or in combination: dimethylsulfoxide, 1,2-ethanediol, 1,2-propanediol, glycerol, 1,2-butenediol, 1,3-butanediol, 2,3-butanediol, formamide, urea, acetamide, hydroxyurea, N-methyl formamide. In further embodiments, the non-permeable solutes can include any of the following components, either singly or in combination: glucose, sucrose, galactose, fructose, trehalose, raffinose, ficol, polyethylene glycol, polyvinylpyrrolidone, polyvinyl alcohol. Where the non-permeable solute is polyethylene glycol, the PG can have an average molecular weight anywhere between 200 and 10,000. Furthermore, the polyvinyl alcohol can have an average molecular weight anywhere between 30,000 and 100,000. Similarly, where the non-permeable solute is polyvinylpyrrolidone, that solute can have an average molecular weight anywhere between 10,000 and 360,000.

### EXAMPLE 1

Optimization of vitrification using the methods disclosed herein has been conducted for metaphase II (MII) human oocytes using a solution containing ethylene glycol as the permeating solute and sucrose as the non-permeating solute (the base solution consisted of saline supplemented with these two solutes).

### Step One

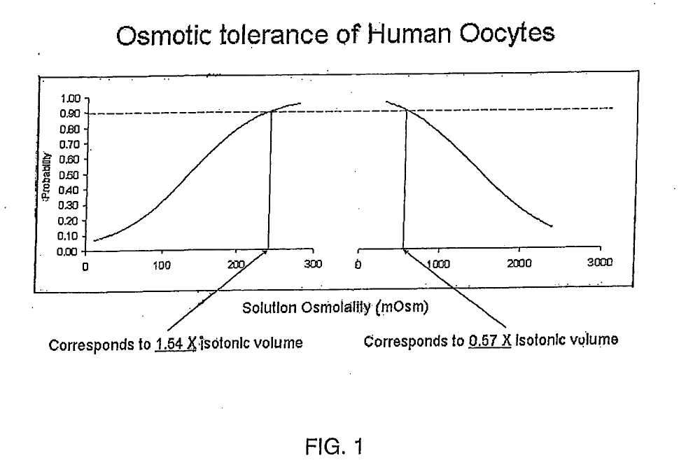

The first step (determining the osmotic tolerance of cells as measured by their cell volume changes) was conducted by incubating oocytes in solutions of various concentrations of sucrose and determining the effect on the MII spindle within the cell. (See, e.g., Mullen S F, Agca Y, Broermann D C, Jenkins C L, Johnson C A and Critser J K (2004) “The effect of osmotic stress on the metaphase II spindle of human oocytes, and the relevance to cryopreservation”, Hum Reprod 19, 1148-1154). Then, a tolerable range of osmolalities was chosen as reflected in FIG. 1. It is noted that the upper curves in FIG. 1 represents the upper limit to the 95% confidence interval for the man probability of disruption of the MII spindle in MII human oocytes. In this example, a 90% expected survival was selected as the limit for osmotic damage, as represented by the vertical lines in FIG. 1. Thx-axis osmolality values corresponding to the 90% survival lines led to a calculated solution osmolality range of 57-154% of the isotonic volume, in which the Boyle van't Hoff relationship for human oocytes was used for the calculation, as described in “Osmotically inactive volume, hydraulic conductivity, and permeability to dimethyl sulphoxide of human mature oocytes”, J Reprod Fertil 117, 27-33 (Newton H, Pegg D E, Barrass R and Gosden R G (1999)), the disclosure of which is incorporated herein by reference.

### Step Two

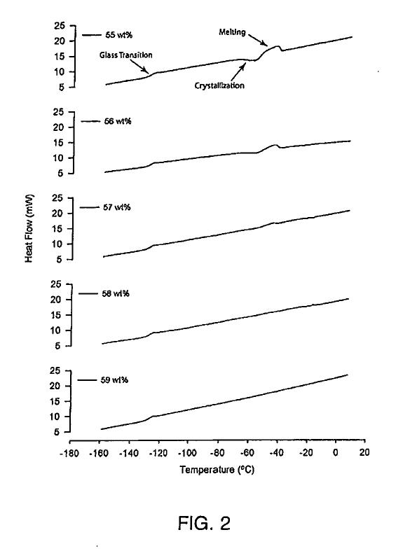

In accordance with the present invention the second step is to determine an optimal combination of permeating and non-permeating solutes to maintain a vitrified state. In this example, this step was conducted for solutions containing sucrose (at concentrations ranging from 0.1 to 1.1 molal) and ethylene glycol using differential scanning calorimetry. FIG. 2 shows results for solutions of various total solute concentrations (in weight %) when sucrose is held at 0.3 molal. FIG. 2 includes thermograms for these solutions, based on cooling rates of 100° C./min and warming rates of 10° C./min. The uppermost thermogram shows crystallization and melting peaks for 55% weight, but at 59 weight % there is no evidence of crystallization and melting during warming. A similar analysis was conducted for solutions containing sucrose at 0.1, 0.5, 0.7, 0.9, and 1.1 molal.

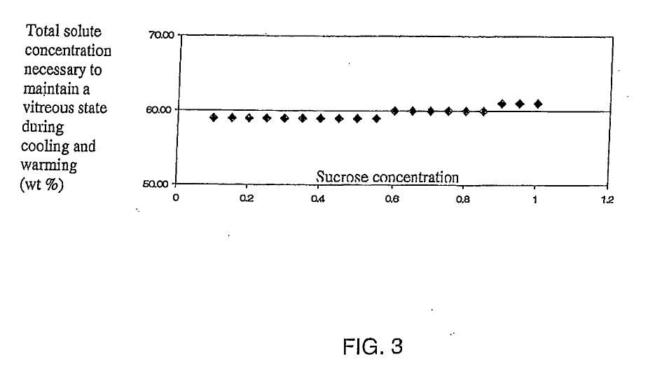

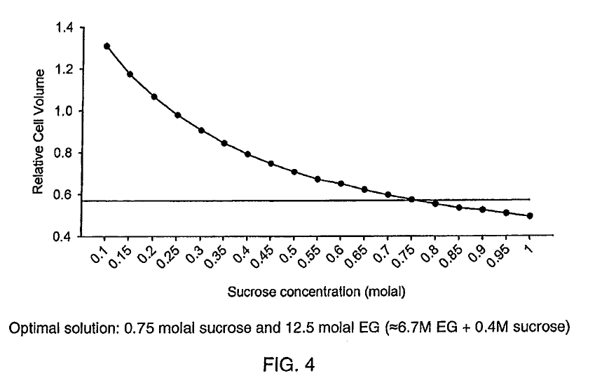

FIG. 3 shows the relationship between the total solution concentrations necessary to maintain a vitreous state and the sucrose concentration in the solution ranging from 0.1 to 1 molal. FIG. 4 shows the effect of incubating MII human oocytes in such solutions on the equilibrium cell volume. This graph in FIG. 4 thus shows the effect on cell volume of vitrification solutions containing different concentrations of sucrose having a total concentration of sucrose and EG in saline necessary to maintain a vitreous state during cooling and warming. The optimal solution in this example is at the point at which the cell volume shrinks to the lower limit of the osmotic tolerance range, in this instance 0.57× isotonic volume as indicated in FIG. 1. Thus, the horizontal line in FIG. 4 at a relative cell volume of 0.57 marks the lower cell volume tolerance (lower osmotic tolerance). The intersection of this horizontal line with the concentration curve thus identifies an optimal solution as sucrose at 0.75 molal and EG at 12.5 molal.

### Step Three

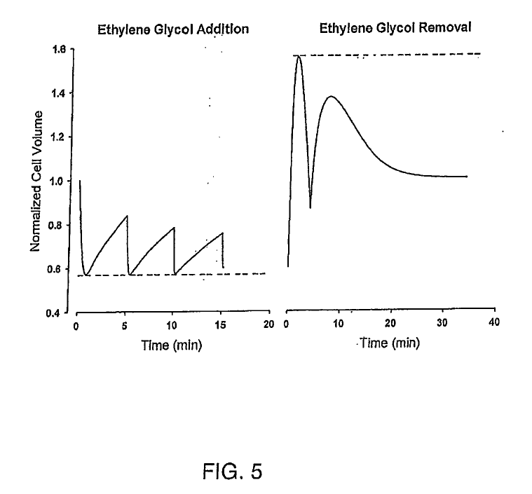

The third step of the present invention involves determining a means to add and remove the permeating cryoprotectant from the cells in question without exceeding osmotic tolerance volume limits of the cells, as defined in Step 1. The results from this analysis are presented in FIGS. 5-7. The volume changes associated with a proposed stepwise method for adding and removing EG to and from a MII human oocyte is depicted in FIG. 5. The horizontal dashed lines represent the volume tolerance limits within which the cells were kept to avoid osmotic damage to the cells.

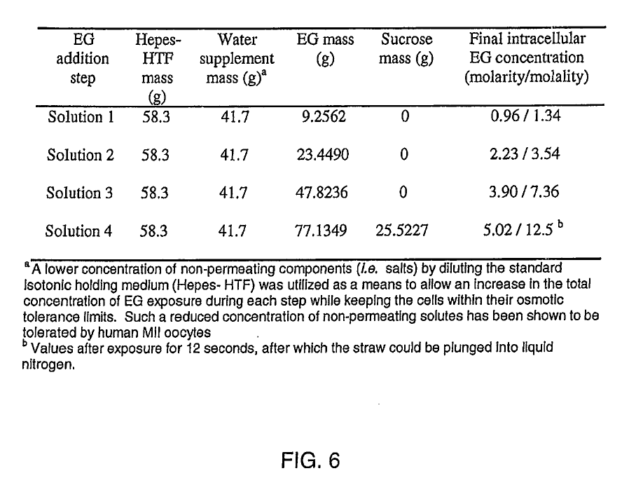

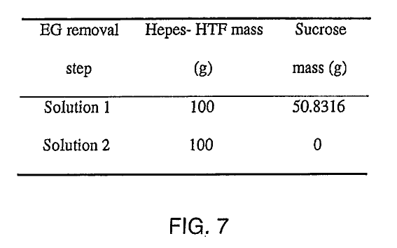

In this example, a 4-step ethylene glycol addition procedure was determined to fit the criteria. (It should be noted that more or fewer steps could be employed with corresponding changes in the concentrations of the solutes). The solution parameters for this 4-step process for the addition of EG to MII human oocytes are shown in the table of FIG. 6. In the present example, each of the first three steps proceeded for 5 minutes at 25° C. The table in FIG. 7 shows the solution parameters for the CPA removal steps. The first of the two steps proceeded for 4 minutes at 25° C.

### EXAMPLE 2

In a further example, a study was conducted to first determine the quantitative permeability of mature human oocytes to ethyleneglycol (EG) and to water in the presence of EG at different temperatures. The study further assesses the relationship between the amount of EG and sucrose in saline necessary to maintain an ice-free state when cooling to and warming from cryogenic temperatures. The study finally implemented known computer modeling techniques to investigate vitrification methods based upon the experimental results.

The study of this Example 2 thus proceeded as follows:

### Experiment 1: Oocyte Permeability to Ethylene Glycol Oocyte Collection

All chemicals were purchased from Sigma-Aldrich (St. Louis, Mo.) unless otherwise stated.

This study design was approved by the Shandong Provincial Hospital Institutional Review Board. Informed, written consent was obtained from all patients who donated oocytes to this study. The average age of the oocyte donors was 28 years (range: 22-33 years; n=19). Cumulus-oocyte complexes (COCs) derived from smaller follicles of the total follicle cohort were used in this study. Only oocytes having a normal appearance and a visible first polar body were used in this experiment. The ovarian stimulation protocol commenced with 150 IU human menopausal gonodotropin (HMG; Iebaode, Iizhu, China) by muscle injection from menstrual cycle day 5.

Thirty-six hours after an injection of 10,000 IU human chorionic gonadotropin (hCG; Iizhu, China), COCs were aspirated transvaginally. The COCs selected for the study were separated from the others and placed into 4-well dishes containing human tubal fluid medium (HTF; Irvine Scientific)+10% serum substitute supplement (SSS; Irvine Scientific) in a culture incubator at 37° C. with a 5% CO₂/air atmosphere. The cumulus cells were removed approximately 3-4 hours after collection using hyaluronidase (Sigma-Aldrich, St Louis Mo. USA; 80 IU/ml) and gentle pipetting. Oocytes were returned to HTF after cumulus stripping. The oocytes remained in culture for no more than 5-6 hours prior to use in the experiment.

### Oocyte Perfusion and Image Acquisition

Measurements of oocyte permeability were conducted at different temperatures on an inverted microscope (Leica DMIRB, China). Perfusion of the oocytes was performed as previously described and validated in the laboratory Oocytes were placed in 20 μl drops of Hepes-HTF (Irvine Scientific) with 10% SSS in a petri dish covered with mineral oil (Vitrolife; Englewood, Colo., USA) to prevent evaporation. An initial photograph was taken of the oocyte in order to calculate the initial volume. Then, approximately 3.5 ml of Hepes-HTF diluted with EG at a final concentration of 1.0 M was added to the dish. When the perfusate merged with the 20 μl drop of base medium, photographs of the oocytes were taken at regular intervals.

The oocytes were held in place during the experiment using a glass holding pipette (Humagen Holding MPH-MED-30, I.D.-20 μm, O.D.-95˜120 μm) and micromanipulators (#RI MODEL SAS11/2-E, Integra Research Instruments, UK). For temperatures lower than ambient, a 35 mm Petri dish (Falcon 35-1008) was placed in an aluminum chamber that was cooled with circulating liquid via a cooling bath (Fisher model 9109) and pumped through the aluminum chamber using polymer tubing. The bottom of the dish was wrapped in aluminum foil and a square hole was cut in the foil to facilitate viewing the oocyte. This was performed so the dish would fit snugly in the chamber to facilitate heat transfer from the chamber to the dish. The temperature in the cooling bath was set to either −2° C. or +7° C. for the 2 cold temperatures. The actual temperature of the media in the dish was warmer than this due to heat exchange of the fluid as it circulated through the tubing and chamber.

For the third treatment, oocyte permeability was measured at ambient temperature (˜25° C.) where the perfusion took place in a 50 mm Petri dish (Falcon 35-1006) placed on the microscope stage with no heating or cooling. Finally, the warmest temperature was attained in a similar fashion but the temperature of the media was raised by using a microscope stage warmer set to 39° C. Variations in temperature of the media occurred due to day-to-day fluctuations in temperature in the laboratory. However, the actual temperature of the media for each replicate during the experiment was measured using a type-T (copper/constantan) thermocouple and an electronic thermometer (model 51 II, Fluke Corporation, Everett, Wash. USA).

After the experimental run, the oocyte was released from the holding pipette on the micromanipulator and the temperature of the medium was measured such that the thermocouple was visible in the microscope field of view, ensuring that the temperature was recorded at the exact location of the oocyte. On several occasions the media was measured both during and after the experiment, and the initial and final temperatures did not vary by more than 0.5° C. The media was either pre-warmed or pre-cooled prior to perfusion, depending upon the experimental conditions. For the experiments using the warm stage, photographs were taken every 3 seconds for the duration of the oocyte volume excursion. For the experiments at ambient temperature, photos were taken every 5 seconds. For the experiments when the cooling bath was set to 7° C., photographs were taken every 5 seconds initially, and after the oocyte reached a nadir in volume and began to swell, the time duration was changed to 30 seconds. For the coldest temperature, photos were taken every 10 seconds initially, and every 60 seconds during swelling. The exact time of these transitions was recorded during each replication and was accounted for during the calculations to estimate the permeability parameters.

### Image Analysis, Parameter Estimation, and Cell Dynamic Modeling

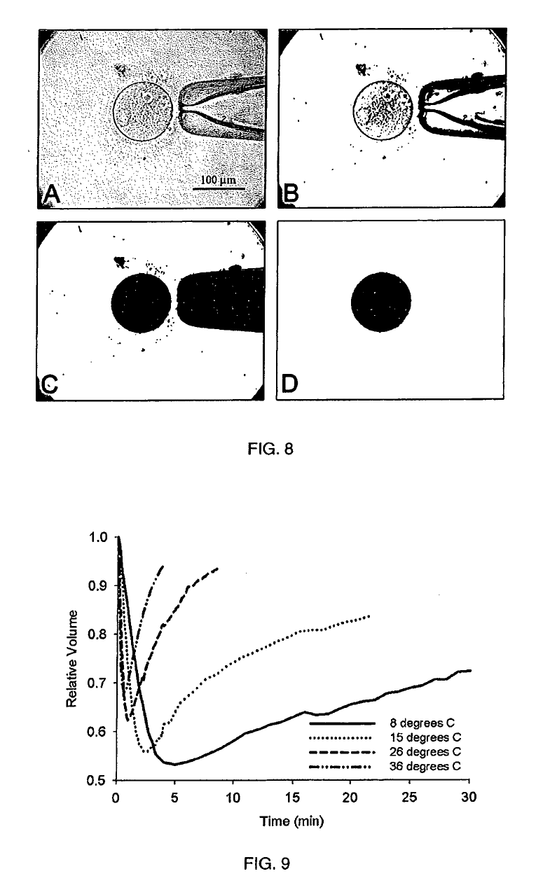

Only those oocytes remaining nearly spherical during the volume excursions were used to estimate the permeability parameters. Forty three of 72 oocytes fit this criterion (˜60%). In several of the instances where the oocytes were rejected for analysis, the cells shrank close to spherical, but folds in the membranes prevented accurate determination of the cell volume. Image analysis was performed using image analysis software (Fovea Pro®, Reindeer Graphics, Asheville N.C., USA, and Photoshop®, Adobe Systems, Inc. San Jose, Calif., USA). To determine the cell volume in each image, the following process was performed, as depicted in FIGS. 8A-D. Initially, the image was thresholded by grey scale to isolate the oolemma, as it is noticeably darker than the area in its immediate surroundings (FIGS. 8A and 8B). Imperfections in this step were manually corrected. The next step involved filling in areas completely surrounded by black pixels (FIG. 8C). The final step involved isolating the oocyte from the remainder of the features in the image by deleting objects other than the oocyte (FIG. 81D). Finally, the area of the oocyte was calculated by counting the number of black pixels, and the diameter of a circle with this equivalent area was calculated. All of these processes were performed by the software, and the software was calibrated with an image of a stage micrometer. The volume of the oocyte was calculated from this diameter assuming spherical geometry.

For this study, a 2-parameter model was used to describe the cell dynamics. The 2-parameter model was adopted versus a 3-parameter model in part because it has been argued that this model is more parsimonious and many previous investigations into membrane permeability of cells including oocytes have shown that the interaction factor (σ) is insignificant. This model uses a pair of coupled, linear ordinary differential equations to describe the change in cell water volume and moles of intracellular permeating solute (e.g. EG). Equation 1 below describes the change in cell water volume (V_{w}) over time (t) as a function of the hydraulic conductivity (Lₚ), surface area (A), gas constant (R: 0.082 L Atm mol⁻¹ K⁻¹), temperature (T, in K), intracellular permeating (m^{e}ₛ) and non-permeating (m^{e}ₙ) solute concentration in osmoles, and the extracellular permeating and non-permeating solute concentration (osmoles of solute (nⁱₛ and nⁱₙ respectively)/cell water volume (V_{w})):

$$\frac{d {V}_{w}}{d t} = - {L}_{p} ART ( {M}_{s}^{e} + {M}_{n}^{e} - \frac{{n}_{s}^{i}}{{V}_{w}} - \frac{{n}_{n}^{i}}{{V}_{w}} ) . \qquad ( 1 )$$

Equation 2 describes the change in intracellular moles of permeating solute (nⁱₛ) over time as a function of the solute permeability (Pₛ):

$$\frac{d {n}_{s}^{i}}{d t} = {P}_{s} A ( {M}_{s}^{e} - \frac{{n}_{s}^{i}}{{V}_{w}} ) . \qquad ( 2 )$$

A spreadsheet was created to estimate the permeability parameters using the Solver tool in Microsoft Excel® (Microsoft, Redmond Wash., USA). Briefly, for each experimental run, the volume change data from the image analysis was imported into the spreadsheet and compared to the theoretical model of volume change data calculated from the above equations. The initial volume of cell water in the oocyte was determined by subtracting the osmotically inactive fraction of the cell volume (0.19× isotonic volume) from the total cell volume estimated from the initial image of the oocyte. Similarly, the total cell volume from the model calculations was determined by summing the cell water volume calculated from Equation 1, the volume of EG in the cell (calculated as the product of the partial molar volume of EG (0.056 L mol⁻¹) and the intracellular moles of EG from Equation 2) and the osmotically inactive cell volume as described above. The Solver tool in Excel® was used to estimate the permeability parameter values by minimizing the sum of squared errors between the experimental cell volume and the volume calculated from equations 1 and 2 for each run. Once these parameters have been estimated, they can be applied in Equations 1 and 2 to calculate the change in volume and intracellular CPA concentration an oocyte will undergo for any CPA addition and removal procedure.

The activation energy for Lₚ and Pₛ was determined assuming an Arrhenius relationship between the parameter and temperature. This relationship can be described as in Equation 3 for Lₚ:

Lₚ=Lₚ₀·e^{-Ea/R(T-T0)}, (3)

where Lₚ₀ and T₀ are reference parameters (e.g. Lₚ at a specific temperature T₀). When 1000/RT (on the abscissa) is plotted against In(Lₚ) (on the ordinate), the slope of the linear regression through these data gives the value for −Eₐ. In this instance, the appropriate value for R is 1.987 cal mol⁻¹ K⁻¹. The equation describing Eₐ of Pₛ is similar, with Pₛ substituted for Lₚ in (3).

### Experimental Design and Statistical Analysis

An incomplete randomized block design was used in this experiment, the blocking factor being a patient. Each oocyte was randomly assigned to one of the treatments. The order of the four temperatures on each day was randomized for each replicate. All randomization procedures were conducted using the random number generator in Excel®. Linear regression analysis was performed with the statistical analysis system (SAS®, Cary N.C., USA). A total of 43 oocytes were analyzed for this experiment. For the four experimental treatments (˜33, 26, 14, and 9° C.), 13, 13, 9, and 8 oocytes were analyzed, respectively.

Experiment 2: Assessing the Amount of EG Necessary to Achieve and Maintain a Vitreous State during Cooling and Warming for Different Concentrations of Sucrose in Saline

This experiment was designed to determine the amount of EG necessary to maintain an ice-free (i.e. vitreous) state during cooling to cryogenic temperatures (˜160° C.; below the glass transition temperature of these solutions) and warming, when the sucrose concentration ranged from 0.1 to 1.1 molal (mol/kg; equivalent to 0.053 to 0.53 molar (mol/L)). In studies of the physical properties of solutions, components of the solutions are usually measured by weight, not volume. A primary reason for this is because weight (in comparison to volume) does not change with temperature. Furthermore, concentrations are usually reported in weight fractions (weight percent (w/w, or wt %)). These conventions were used in this experiment and references was made to the equivalent molar concentrations when comparing the results to previously published studies.

A Diamond Differential Scanning Calorimeter (DSC; Perkin-Elmer Waltham, Mass., USA) outfitted with an autosampler and CryoFill liquid nitrogen cooling system was employed for the thermal analysis. The solutions were composed of water (5 g) and NaCl (0.0045 g) to which various amounts of sucrose and EG were added. For this experiment, spectrophotometric grade (>99%) EG was utilized. All components were weighed on an analytical balance. Errors between the target and actual weight percent were less than 0.1% in all instances. To test the vitrification properties of the solutions, 5 μl (˜0.0055 grams) were loaded into standard 10 μl aluminum DSC pans (Perkin-Elmer Part # BO14-3015), and the pans were hermetically sealed. Embryo-grade water and HPLC-grade (>99.9% purity) cyclohexane were also run as calibration standards.

Analyses were conducted at a cooling rate of 100° C./min and a warming rate of 10° C./min to be consistent with previous studies. In instances when crystallization and melting peaks were not clearly evident on the curves, plots of the heat flow as a function of temperature were analyzed by fitting a polynomial curve to the data and determining if a crystallization or melting peak was evident above the random noise of the signal. Random noise was estimated by determining the standard deviation of the actual signal from the expected value from the polynomial fit. When an apparent crystallization peak followed by a melting peak in the expected temperature range was noted, and the maximum point on the melting peak was beyond three standard deviations from the expected value, it was classified as a thermal event. Only when three independent solutions confirmed the absence of crystallization and melting was the solution classified as having achieved and maintained a vitreous state throughout the cooling and warming procedure.

Experiment 3: Computer Modeling toward an Optimal Vitrification Method for Human Oocytes

Using the experimental data and osmotic tolerance data from the prior studies, methods were investigated to vitrify human oocytes using computer modeling. One goal was to develop a method which should ensure ice-free cryopreservation (i.e. achieve vitrification and preclude devitrification during cooling and warming) and prevent osmotic damage to the cell during the CPA addition and removal processes using an optimal combination of sucrose and EG.

The first step was to determine the appropriate composition of a vitrification solution using EG and sucrose as the cryoprotectants. There were two criteria used to make this determination: (1) the total solute concentration should be high enough to preclude ice formation during cooling and warming at rates applicable to devices used for cryopreservation currently in practice; and (2) the sucrose concentration should be as high as possible (high enough just to reach the oocyte osmotic tolerance threshold, which will allow the lowest amount of EG to be used, reducing the chemically-toxic properties of the solution).

It is known that the concentration of solutes necessary to achieve vitrification and avoid devitrification decreases with increasing cooling and warming rates. Therefore, it was necessary to account for this property when estimating the appropriate solution composition. Prior studies have confirmed that warming rates are more critical than cooling rates when trying to maintain a vitreous state. Therefore, the focus of the present experiment was on the warming rates necessary to avoid devitrification from this point forward. In the prior studies it was determined that a solution containing 50 wt % EG should maintain a vitreous state when the warming rate is on the order of 1×10³° C./min, and 48 wt % when the warming rate is on the order of 1×10⁴° C./min. It has previously been shown that 59 wt % EG is necessary to maintain a vitreous state during cooling and warming at the rates that were used in the DSC analysis. Cooling and warming rates using standard ¼ cc straws and the so-called ultra-rapid cooling devices (e.g. cryotops, open-pulled straws) can achieve cooling and warming rates on the order of 1×10³ and 1×10⁴° C./min, respectively. Because the difference in wt % necessary to avoid devitrification with a warming rate of ˜1×10⁴° C./min compared to 1×10³° C./min is relatively minor (48 vs. 50 wt %), the lower warming rate was applied, applicable to ¼ cc straws for the remainder of the analysis (please see below for a more complete discussion of this choice). This analysis suggests that the weight percent of solutes can be reduced by 15% (59 vs. 50 wt %) when the warming rates are increased from ˜1×10¹ to ˜1×10³° C./min. Therefore, the investigation of an ideal vitrification solution focused on solutions across the range of sucrose molalities used in Experiment 2 above, yet having a total solute composition reduced by 15 wt %.

For the next step, a calculation was made as to the degree to which solutions containing the various concentrations of EG and sucrose from the previous step will affect the equilibrium volume of a human oocyte. This was done by solving Equations 1 and 2 above using the appropriate values of the variables for each solution. For the modeling it was assumed that the oocyte would have properties of an average human oocyte, namely a cell radius of 63 μm, a permeability to water of 0.69 μm/min/atm, and a permeability to EG of 9.16 μm/min (average values at 25° C. from the present work).

From this analysis a solution was determined that would have the optimal combination of EG and sucrose for vitrification, meaning that it would have the maximum amount of sucrose, and hence the minimum amount of EG, yet would not result in the oocyte exceeding the estimated osmotic tolerance. Osmotic tolerance data for human oocytes are relatively scarce compared to data for oocytes from other taxa. In a prior study, the relationship was determined between osmolality and the morphology of the MII spindle as an experimental endpoint using a logistic regression analysis. Using this data, the 95% confidence interval for the mean was calculated using the logistic regression model. The osmotic tolerance was then defined as the range of osmolalities which 90% of oocytes should tolerate, and then estimated the corresponding cell volume utilizing the previously published Boyle van't Hoff relationship for human oocytes. The results from this analysis suggest that 90% of human oocytes should tolerate changes in cell volume ranging from 57% to 154% of their isotonic volume. Thus, of the solutions analyzed, the chosen solution contained a combination of sucrose and EG such that the cell volume would be reduced to 57% of the isotonic volume upon equilibration.

Finally, after determining the optimal solution using these criteria, a model was generated of EG addition and removal procedures which will keep the oocytes within the osmotic tolerance limits defined above. Cell dynamic modeling was performed using Mathematica® (Wolfram Research, Champaign Ill., USA).

For the cell dynamic modeling carried out in the experiments described above, several simplifying assumptions were used as commonly employed in previous cryobiology studies. These include: 1) the extracellular space is assumed to be infinite and the intracellular space is the volume of a sphere (V=4πr³/3); 2) the surface area of the cell is constant and determined by the initial cell radius (A=4πr²); 3) the intracellular and extracellular solutions are assumed to be ideal and dilute; 4) the hydrostatic pressure across the cell membrane is assumed to be zero. It was also assumed that the osmotic coefficients for the solutes were equal to 1, such that molalities and osmolalities are equivalent, and the additivity of solute osmolalities are linear.

### Results

### Experiment 1 Results: Oocyte Permeability to Ethylene Glycol

When the oocytes were abruptly exposed to a solution containing 1.0 mol/L EG, they responded osmotically, first by shrinking, then swelling, as expected. Furthermore, the degree of shrinkage and time for swelling was influenced by the temperature of the solution, as shown in FIG. 9. In particular, as demonstrated by the curves in FIG. 9, lower temperatures are associated with a greater total volume loss and a slower return to isotonic volume. This response is dictated by the permeability of the oolemma to both water and EG, as described by Equations 1 and 2 above. The average radius of human oocytes in an isotonic solution is known to be 63 μm. The mean radius of human oocytes analyzed in this study was nearly identical (63.7 μm). The population was slightly skewed toward higher values, and this is reflected by a slight decrease in the median value (63.4 μm).

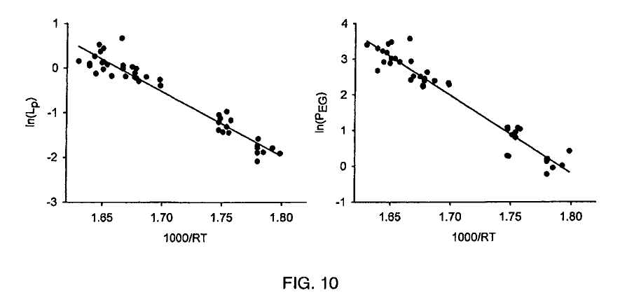

The relationship between temperature and the two permeability parameters for the oocytes in the present study is illustrated in FIG. 10. As expected, temperature and permeability were strongly correlated (r=−0.97 for both Lₚ and Pₛ). The values of Lₚ differed by an order of magnitude across the temperature range (from 0.15 μm/(min·atm) at 6.7° C. to 1.17 μm/(min·atm) at 35.7° C.). For the four experimental treatments (˜33, 26, 14, and 9° C.), the coefficients of variation for Lₚ were 21%, 34%, 17%, and 15%, respectively. For Lₚ, the activation energy was 14.42 Kcal/mol (95% confidence interval: 13.19 to 15.65 Kcal/mol). The values of Pₛ also differed by an order of magnitude across the temperature range in the present study (from 1.5 μm/min at 6.7° C. to 30.0 μm/min at 35.7° C.). For the four experimental treatments (˜33, 26, 14, and 9° C.), the coefficients of variation for Pₛ were 24%, 22%, 27%, and 19%, respectively. For Pₛ, the activation energy was 21.20 Kcal/mol (95% confidence interval: 19.49 to 22.91 Kcal/mol). Linear regression allows the calculation of the expected value of these parameters at any temperature T (in K). In this instance, Lₚ can be calculated by solving the following equation:

Lₚ=Exp[−14420/(1.987T)+23.983] (4)

and Ps can be calculated by solving the following equation:

Pₛ=Exp[−21780/(1.987T)+38.998]. (5)

Experiment 2 Results: Assessing the Amount of EG Necessary to Achieve and Maintain a Vitreous State during Cooling and Warming for Different Concentrations of Sucrose

Examples of DSC thermograms during warming for solutions containing 0.3 m sucrose with varying total solute concentrations are shown in FIG. 2. The thermal transition on the far left of the curves, near −130° C., represents the glass transition. Two other transitions are evident as the solution warms and approaches −60° C. The first change, when the heat flow decreases, represents the heat of fusion of water as ice crystallization occurs. Approximately 20° C. warmer, another transition occurs, as the ice crystals melt. It is evident that the magnitude of these later transitions becomes smaller as the total weight percent increases, until no evidence for crystallization or melting is apparent. In all of the solutions tested, there was no evidence for crystallization during cooling (data not shown). The total concentration of the solution necessary to prevent crystallization during warming increased as the sucrose concentration increased from 0.1 to 1.1 mol/kg; solutions containing 0.1, 0.3, 0.5, 0.7, 0.9, and 1.1 mol/kg sucrose required 59, 59, 59, 60, 61, and 61 wt %, respectively.

Experiment 3 Results: Computer Modeling toward an Optimal Vitrification Method

Having established the permeability of human oocytes to EG and water in the presence of EG in Experiment 1 and the appropriate proportions of EG and sucrose to include in vitrification solutions in Experiment 2, the effect of solutions was calculated with the same proportions, yet with a total concentration reduced by 15%, on the equilibrium volume of human oocytes. As expected, the volumes that human oocytes attained upon equilibration were reduced in direct proportion to the sucrose concentration, as shown in FIG. 4. The equilibrium volume was calculated by solving Equations 1 and 2 above using the concentrations of permeable (EG) and nonpermeable (sodium chloride and sucrose) solutes in the respective solutions. As the sucrose concentration is increased, the amount of EG in the solution necessary to achieve and maintain a vitreous state during cooling and warming decreases. However, as the sucrose concentration increases, a greater effect on the cell volume will occur, as shown in FIG. 4. In accordance with the methods disclosed herein, the optimal solution was chosen as a combination of EG and sucrose such that the solution would have the maximum amount of sucrose (reducing the EG concentration) yet would not result in excessive cell shrinkage.

From this analysis it was determined that a solution containing 0.75 mol/kg (0.40 mol/L) sucrose would dehydrate the cell just to the point of the osmotic tolerance threshold (57% of the isosmotic volume), as represented by the horizontal line in FIG. 4. According to the results from the second experiment, the concentration of EG necessary to maintain a vitreous state during cooling and warming in a solution containing 0.75 mol/kg sucrose is 12.49 mol/kg (6.72 mol/L). Thus, this combination was chosen as the optimal solution for further analysis.

An appropriate stepwise method was used to expose human oocytes to EG with this optimal solution as the final target, in preparation for vitrification. Sucrose is only necessary in the final vitrification solution. Having sucrose in the initial solutions may be less efficient because it would cause osmotic shrinkage of the cell and reduce the amount of EG in the initial solutions to which oocytes can safely be exposed (safely in this context refers to preventing osmotic damage). Using Equations 1 and 2 described above, and the osmotic tolerance limits discussed, an optimal EG addition and removal procedure for human oocytes was determined. The procedure requires a 4-step CPA addition (5 minutes for the first 3 steps) and a 2-step CPA removal. The details of this procedure can be found in the tables in FIGS. 6 and 7.

Equilibrium freezing (i.e. slow-cooling) is the predominant method in practice for human oocyte cryopreservation. Despite approximately twenty years of effort, the results from this approach remain highly variable, and human oocyte cryopreservation is still considered an experimental procedure. The relatively poor performance of equilibrium freezing is known. In recent years, vitrification methods have been applied to cryopreserve human oocytes to determine if improvements can be made.

### Ethylene Glycol Permeability and its Use for Vitrification

Ethylene glycol is one of the primary permeating cryoprotectants used in vitrification methods, principally due to its relatively low toxicity compared to other compounds. Because vitrification procedures necessitate the use of high solute concentrations, toxic and osmotic damage is more likely. Concern about osmotic damage is particularly important when using EG, as mammalian oocytes generally have a lower permeability to EG relative to other permeating CPAs. For example, it has been shown that the oolemma in mouse oocytes has a lower permeability to EG relative to PG and DMSO. In a 1999 study the average permeability to EG was 0.24 μm/s at 30° C., which is similar to the estimated average permeability of human oocytes at this temperature obtained from the present study (0.27 μm/s). In contrast, the permeability of Rhesus macaque oocytes to EG at this temperature was reported to be much lower (0.14 μm/s;. Volumetric response of the oocytes in prior studies suggests that only glycerol had a permeability value lower than EG.

The results from the present study also show that in vivo matured MII human oocytes have a lower permeability to EG in comparison to PG and DMSO. At 24° C., the average permeability values for the three CPAs are 8.2, 15.0, and 16.8 μm/min, respectively. The effect of this difference on the volume response of human oocytes to cryoprotectant solutions can be calculated using Equations 1 and 2 from above. For example, a single-step exposure to 1.5 mol/L PG, DMSO, and EG at 24° C. will result in the reduction of cell volume to approximately 67%, 61%, and 53% of the isosmotic volume for the respective cryoprotectants. Although the difference in cell volume response suggests that EG may be an inferior permeating agent for cryopreservation of human oocytes, the volume changes associated with permeating cryoprotectants are only one of many important factors to consider when designing cryopreservation procedures. Because volume changes can be modulated by changing the method used to expose the cells to such compounds, the inherent toxicity of the permeating cryoprotectant may be a more important consideration. The average permeability of oocytes to EG at 30, 22, and 8° C. has been recently reported to be 28.5, 11.7, and 3.7 μm/min. Overall, these permeability values are higher than the expected average permeability of in vivo matured human oocytes at the respective temperatures as determined from the present study (17.1, 6.4, and 1.0 μm/min). This difference suggests that an optimal cryopreservation protocol for in vitro matured human oocytes will differ from one developed for in vivo matured oocytes.

Osmotic stress, and the resulting cell volume excursions, is one of the primary theories of injury during cryopreservation. Previous studies on the effects on bovine oocytes of exposures to anisosmotic solutions has shown that exposure to concentrations of 1200 milliosmolal or greater (equivalent to a reduction in volume to ˜40% of the isotonic volume (determined from the analysis of)) reduced the potential to develop to blastocysts by ˜50% relative to untreated cells. Rhesus macaque oocytes suffered membrane damage after exposure to concentrations of EG at 3 mol/L or higher using a single-step addition and removal, although it was not determined if this damage occurred specifically as a result of the volume excursions, or from a chemical effect of the EG, or an interaction between these two factors. While it has been shown that human oocytes can tolerate exposure to fairly high concentrations of mono- and disaccharides (up to 1.5 mol/L; equivalent to a reduction in volume to ˜35% of isotonic) as measured by immediate viability using membrane integrity and enzyme activity, a reduced tolerance was reported as measured by MII spindle morphology.

### Identifying Optimal Combinations of EG and Sucrose for Vitrification

When designing a vitrification procedure, one consideration is whether the cryoprotectant solution can form and maintain a stable glass during cooling and warming. Additionally, the lowest concentration of solutes necessary to meet this condition should be used to minimize the potential toxicity of the solution. Because solutes have different physical properties, the relative amount of each solute in a solution will affect the ability of a solution to vitrify. Ethylene glycol and sucrose are commonly used as permeating and non-permeating solutes for vitrification of mammalian oocytes, yet little is known about the glass-forming properties of aqueous solutions containing these solutes. A prior analysis similar to the one undertaken in this study, but with fewer concentrations of sucrose (0, 0.1, 0.5, and 1.0 mol/kg) produced similar results in determining that the solutions required 59, 60, 61, and 65 wt % at the respective sucrose concentrations to maintain a vitreous state during cooling and warming. In both the prior and present studies, the total weight percent necessary to avoid crystallization increased as EG was replaced with sucrose. This result is expected, as aqueous solutions containing only sucrose as the solute require higher concentrations (>70 wt %) to maintain a vitreous state during slow warming compared to solutions containing EG as the only solute (˜59 wt %).

Investigating Methods for Vitrification Using Computer Modeling Based upon Fundamental Principles

Cellular damage can occur during any one of the steps in a cryopreservation procedure. Optimizing such a multi-step procedure through purely empirical means would require a very large experiment—the number of treatment combinations could easily be in the thousands. By using fundamental principles and computer modeling, estimates of optimal methods can be arrived at through rational analysis, with the potential to save a significant amount of resources by narrowing the choices to test via empirical methods. Such an approach is particularly appealing for human oocyte cryopreservation, where experimental material is scarce, and experimental design is significantly influenced by ethical considerations. The present study uses such principles to make an initial prediction of an optimal vitrification method for human oocytes using EG and sucrose as cryoprotectants. These predictions were based on several criteria, including the necessity of attaining a stable vitreous state during cooling and warming, using the minimum amount of permeating cryoprotectant, and designing the method to reduce the potential for osmotic damage to the cell.

The reasons for moving away from equilibrium freezing methods and toward the use of vitrification include reducing cell damage associated with chilling injury and ice formation. If the latter is a true goal, then one should use a solution that maintains a stable vitreous state during cooling and warming. However, using solutions with higher concentrations of solutes than is necessary to maintain a vitreous state exposes cells to unnecessary risks of chemical and osmotic damage. The modeling of the present study used the measured vitrification properties of solutions from the second experiment as a guide for determining the appropriate concentration of the various solutes. If solutions are used that are not true vitrification solutions, the degree to which ice forms and the size of the resulting crystals are difficult to control. Having such little control over an important variable such as ice formation is likely to add to the variability of a method.

Vitrification methods for mammalian oocytes have evolved toward the use of devices to achieve so-called “ultra-rapid cooling” following their application with bovine oocytes. Because of the results from these and similar studies, an implicit assumption seems to have developed in the field of human oocyte cryopreservation that successful vitrification requires cooling rates which can only be attained with such devices. In fact, many prior reports on human oocyte vitrification have used such devices, including open-pulled straws, electron microscope grids cryoloops and cryotops. However, this assumption may not be universally valid. Firstly, the use of different devices in an experimental comparison introduces potentially confounding variables other than cooling and warming rates (e.g. the time for which the cells are exposed to the vitrification solution prior to plunging into liquid nitrogen). Secondly, in particular reference to human oocytes, very high cooling rates may not be necessary because previous studies have suggested that human oocytes are more tolerant to chilling than bovine oocytes. One possible reason for this is the relatively low level of intracellular lipids in human oocytes compared to oocytes from other domestic animals (e.g. cattle and swine), a property which makes these oocytes chilling sensitive.

Current vitrification methods have also evolved to rely upon a very brief exposure to the final vitrification solution, and it is often assumed that the damage from longer exposures is due to chemical toxicity of the solutes. However, such data are equivocal, as the chemical effect is completely confounded by the osmotic effect. Several reports with mammalian oocytes have shown that cryopreservation outcomes can be improved if the osmotic effect of exposure to the solutions is modulated by prolonged CPA addition and/or removal, suggesting that consideration of the osmotic effects is at least as important as the chemical effects.

The modeling disclosed herein focuses on the use of a solution that could maintain a stable vitreous state during cooling and warming when using a standard ¼ cc freezing straw for several reasons. First, there are years of experience in the clinical IVF community with the use of these devices for cryopreservation of other samples. The use of some of the ultra-rapid cooling devices has been reported to be cumbersome, potentially increasing the likelihood of damage to the oocytes during their use, losing oocytes during handling, and requiring extensive technical training periods. In addition, unlike many of the other devices used, standard freezing straws can be safely sealed, thus avoiding the potential for contamination through direct contact with liquid nitrogen. It can be acknowledge that, more recently, some of the ultra-rapid cooling devices have been modified to be fully sealed systems and should alleviate this problem. Furthermore, the design of some of these sealed systems makes it likely that the cooling and warming rates will not be as fast as the open systems.

Although seemingly paradoxical, the larger thermal mass of a ¼ cc straw may have benefits. It is well known that devitrification and recrystallization are serious risks when vitrification solutions are used that are near the threshold of thermodynamic stability. For a device with a low thermal mass, accidental warming, and the concomitant risk of devitrification, is higher than a device with a higher thermal mass. Finally, it is often argued that the higher cooling rates attainable with ultra-rapid cooling devices allow a significant reduction in the concentration of cryoprotectant necessary to maintain stable vitrification (up to 30% according to one recent report). However, other analyses based upon measured physical properties of solutions, suggest that the actual reduction is quite small (˜2%), at least for EG, given the difference in cooling rates between standard freezing straws and the other devices.

Even for bovine oocytes the necessity of ultra-rapid cooling has been successfully challenged. Certain prior studies attempted to refine methods for bovine oocyte vitrification using ¼ cc straws to determine if success rates comparable to those achieved by ultra-rapid cooling methods could be attained. The best results they achieved rivaled those originally achieved with EM grids and open-pulled straws. The method employed in this prior study is similar to what has been proposed to be an optimal method based upon the computer modeling of the present disclosure. In one study, the best results were achieved using a solution called ES40, which contains EG at a concentration of 7.15 mol/L, whereas the optimal solution proposed herein contains EG at 6.72 mol/L. In that prior study the sucrose concentration in ES40 is 0.35 mol/L, whereas in the present solution the sucrose concentration is 0.4 mol/L. A significant improvement in survival was accomplished in the prior study by changing the method for CPA addition to include a prolonged addition procedure (3 steps total vs. 1 or 2 steps). This is very similar to the conclusions derived from the analysis of the present disclosure. Overall, these support the conclusion that osmotic stress is a significant concern when vitrifying mammalian oocytes, yet can be overcome by changing the CPA addition and removal procedures.

Another prior study was conducted to test different vitrification methods directly on human oocytes in which 2 solutions were tested with the cryotop method, one contained 6.8 mol/L EG and the other contained 5.0 mol/L EG. Each solution also contained sucrose at 1.0 mol/L. The proportion of oocytes that showed normal morphology and blastocyst formation was higher in the solution containing the lower concentration of EG. The optimal solution proposed according to the methods of the present disclosure contains EG at 6.72 mol/L, which at first blush may suggest that it will perform poorly compared to a solution with a lower concentration. However, the proposed solution also contains considerably less sucrose (0.4 mol/L), which when coupled with the proposed method for CPA addition and removal should reduce the damaging osmotic effects associated with its use.

There are several aspects of the embodiments of the modeling methods disclosed herein that may influence the predictive accuracy. Much of the model restrictions were focused on the estimates of osmotic tolerance based upon MII spindle morphology. It is recognized that the developmental potential of human oocytes may be a more sensitive indicator of osmotic tolerance than spindle morphology, as has been shown with bovine oocytes. However, to date, for human oocytes this is the best estimate of osmotic tolerance available. In addition, prior experiments on the effect of osmotic stress on human oocytes was conducted in the absence of intracellular cryoprotectants which may have an effect on the spindle microtubules, and hence the estimates of osmotic tolerance.

One of the assumptions in the model is that achieving and maintaining a true vitreous state is a prerequisite for a robust procedure. It is known that cells can survive freezing in the presence of ice (both extracellular and intracellular). Therefore, solutions with lower solute concentrations than the one proposed may allow successful cryopreservation, despite not being able to vitrify. However, the tolerance of cells to ice formation (particularly intracellular ice formation) is not well understood. Furthermore, controlling ice formation and growth is difficult. Therefore, it is believed that, if true vitrification can be achieved and the potential damage from the solution used to achieve vitrification can be managed, preventing ice formation during cooling and warming is preferable.

The embodiments of the modeling methods disclosed herein focused on a vitrification method using a single permeating cryoprotectant (EG). As was stated above, EG is generally less toxic than other compounds. Prior studies have shown that mouse oocyte developmental potential was reduced by only 30% after exposure to 7 mol/L EG in a 2-step manner, and that mouse oocytes could tolerate 8 mol/L EG fairly well if the exposure time was less than 1 minute (mean blastocyst development rate of ˜50% vs. ˜62% for controls), and exposure to 6 mol/L EG for 5 minutes had no effect on development compared to untreated oocytes. A significant reduction in bovine oocyte developmental potential has been found when the cells were exposed to 4 or 5.5 mol/L EG in a single step. However, because the exposure was conducted in a one-step manner, osmotic effects could account for much of the damage. The procedure proposed herein would expose human oocytes to less than 4 mol/L EG throughout the EG loading steps up until transfer to the vitrification solution. The oocytes should reach near equilibrium with the EG after exposure to the final solution within 12 seconds. Thus, the cells need not be exposed to the final solution for long before transfer to liquid nitrogen.

In summary, the methods disclosed herein were initially conducted to examine fundamental cryobiological properties of human oocytes and vitrification solutions in a first step to optimize vitrification methods. The results from applying these methods suggest that successful vitrification in standard freezing straws can be achieved, as the resulting theoretically-optimized method is similar to one shown to improve vitrification with bovine oocytes.

What is claimed is:

1. A method to identify optimal combinations of solutes for inclusion in a vitrification solution and to identify optimal procedures to add and remove such solutes from cells without causing osmotic damage, comprising:
a) determining an optimal combination of solutes in which the combination: i) contains a combination of permeable and non-permeable solutes such that the entire solution will maintain a vitreous state during cooling to cryogenic temperatures (<140 K) and warming from cryogenic temperatures; ii) contains concentrations of permeable solutes that can be tolerated by the cells; iii) contains the maximum amount of non-permeable solutes in relation to permeable solutes such that when the cell is allowed to come to equilibrium with the said solution, the cell volume will not be reduced below a level deemed tolerable to the cell population;
b) determining an optimum method to load the permeable cryoprotectants into and unload the permeable cryoprotectants from the cells in a stepwise manner such that the cells are exposed to a solution containing the permeating cryoprotectants in a concentration that is more dilute than the concentration contained in the solution in which the cells are cooled, wherein the total concentration of the initial solution of a first step will be such that, when the cells are incubated in the solution, the cells will shrink osmotically just to the point of reaching a tolerable volume, and after a predetermined amount of time, the cells are transferred in a second step to a second solution containing the permeable cryoprotectants at a concentration higher than the first solution, but only at a concentration such that when the cells are transferred to the second solution the cells do not shrink below the cell volume deemed tolerable, and in subsequent steps the concentrations of cryoprotectants are increased until the point at which the cells can be transferred to the final solution used to vitrify the cells and the cells will equilibrate with the final solution and not shrink below the volume deemed tolerable.

2. The method of claim 1 where the cells consist of any isolated cell type.

3. The method of claim 1 where the permeable solutes include any of the following components either singly or in combination: dimethylsulfoxide, 1,2-ethanediol, 1,2-propanediol, glycerol, 1,2-butenediol, 1,3-butanediol, 2,3-butanediol, formamide, urea, acetamide, hydroxyurea, N-methyl formamide.

4. The method of claim 1 where the non-permeable solutes include any of the following components, either singly or in combination: glucose, sucrose, galactose, fructose, trehalose, raffinose, ficol, polyethylene glycol, polyvinylpyrrolidone, polyvinyl alcohol.

5. The non-permeating cryoprotectant of claim 3 wherein the said polyethylene glycol has an average molecular weight anywhere between 200 and 10,000.

6. The non-permeating cryoprotectant of claim 3 wherein the said polyvinylpyrrolidone has an average molecular weight anywhere between 10,000 and 360,000.

7. The non-permeating cryoprotectant of claim 3 wherein the said polyvinyl alcohol has an average molecular weight anywhere between 30,000 and 100,000.
